(19) United States
(12) Patent Application Publication
(10) Pub. No.: US 2020/0371056 A1
(43) Pub. Date: Nov. 26, 2020

(54) GAS SENSING DEVICE AND MANUFACTURING METHOD THEREOF

(71) Applicant: Chang Gung University, Taoyuan CIty (TW)

(72) Inventors: CHAO-SUNG LAI, Taoyuan City (TW); CHIA-MING YANG, New Taipei City (TW); TSUNG-CHENG CHEN, Yuanshan Township (TW); YU-CHENG YANG, Taibao City (TW)

(21) Appl. No.: 16/565,232

(22) Filed: Sep. 9, 2019

(30) Foreign Application Priority Data
May 22, 2019 (TW) 108117628

(51) Int. Cl. G01N 27/22; H01L 21/04; H01L 21/02; H01L 29/16; G01N 33/00; G01N 27/12

(52) U.S. Cl. CPC G01N 27/227; H01L 21/0425; H01L 21/0217; H01L 21/0234; H01L 21/02527; G01N 27/125; H01L 29/1608; G01N 33/004; G01N 33/0054; G01N 33/0047; H01L 21/02623

(57) ABSTRACT

A gas sensing device comprises a silicon substrate, an insulating layer, a plasma treatment layer, a metal electrode and a sensing layer. The insulating layer is formed on the silicon substrate. The plasma treatment layer is formed on the insulating layer. The metal electrode is formed on the portion of the plasma treatment layer. The sensing layer is formed on a surface of the metal electrode and the plasma treatment layer. Through plasma treatment for the substrate and printing graphene film on the substrate and the electrode, the adsorption characteristics of gas selection ratio for graphene is improved, and the processing time of the plasma treatment is adjusted to optimize the sensing characteristics.

### TECHNICAL FIELD

The present invention relates to a gas sensing device, and more particularly, to a gas sensing device and manufacturing method thereof which can improve the adsorption characteristics of gas selection ratio for graphene through plasma treatment for the substrate.

### BACKGROUND OF RELATED ARTS

There are many harmful gases in the air, such as carbon monoxide, carbon dioxide, methane and ammonia, etc. At present, many related researches of graphene applied to gas sensor have been proposed. It is known that the fabrication process of resistive gas sensor is to deposit a sensing film on the substrate and then form a structure of metal electrode. In order to improve the gas selection ratio of sensing film (two-dimensional material) in resistive gas sensors, the sensing film is generally modified and doped directly, which results in many defects of the film, and thus increases the resistance value of the film.

In addition, because a single resistive gas sensor does not have gas selectivity, in order to achieve gas selection in the prior art, a gas separation system, such as micro-channel, needs to be installed at the front of the sensor to achieve the purpose of identifying kinds of gases. However, the size of the sensor is too large, which is not conducive to the development of miniaturized sensors.

### SUMMARY

To resolve the drawbacks of the prior arts, the present invention proposes a gas sensing device. Through plasma treatment for the substrate and printing graphene film on the substrate and the electrode, the adsorption characteristics of gas selection ratio for graphene is improved, and the processing time of the plasma treatment is adjusted to optimize the sensing characteristics. Through the array arrangement, the device can sense different kinds of gases.

The present invention proposes a gas sensing device, comprising: a silicon substrate; an insulating layer formed on the silicon substrate; a plasma treatment layer formed on the insulation layer; a metal electrode formed on the plasma treatment layer; and a sensing layer formed on a surface of the plasma treatment layer and the metal electrode.

According to an aspect, the present invention proposes a gas sensing device, comprising: a silicon substrate; an insulating layer formed on the substrate; an array plasma treatment layer having a plural of plasma treatment areas, the array plasma treatment layer is formed on the insulation layer, each of the plural of plasma treatment areas includes: a metal electrode formed on a surface of each of the plural of plasma treatment areas; and a sensing layer formed on a surface of each of the plural of plasma treatment areas and the metal electrode.

According to another aspect, the present invention proposes a manufacturing method of a gas sensing device, comprising: (A) providing a silicon substrate; (B) depositing an insulating material on the silicon substrate to form an insulating layer; (C) performing a halide plasma treatment for the substrate for a period of time by a plasma surface modification to form at least one plasma treatment area on the insulating layer; (D) depositing a metal electrode on a partial surface of each the at least one plasma treatment area; (E) coating a two-dimensional material on each the at least one and the metal electrode to form at least one sensing layer; and (F) forming a sensing area of each at least one sensing layer.

Embodiments of the invention are illustrated by way of example, and not by way of limitation, in the figures of the accompanying drawings in which like reference numerals refer to similar elements.

### BRIEF DESCRIPTION OF THE DRAWINGS

FIG. 1 illustrates a schematic diagram of a gas sensing device according to a preferred embodiment of the present invention.

FIG. 2 illustrates a flow chart of the method for manufacturing the gas sensing device according to a preferred embodiment of the present invention.

FIG. 3 shows a schematic diagram of the substrate of the preferred embodiment of the invention.

FIG. 4 shows a schematic diagram of the insulation layer in a preferred embodiment of the invention.

FIG. 5 shows a schematic diagram of the plasma treatment layer in the preferred embodiment of the invention.

FIG. 6 shows a schematic diagram of the metal electrode structure in the preferred embodiment of the invention.

FIG. 7 shows a schematic diagram of the sensing layer in the preferred embodiment of the invention.

FIG. 8 shows a schematic diagram of the gas sensing device according to a preferred embodiment of the present invention.

FIG. 9 shows the measurement comparison charts based-on different concentration of ammonia and different plasma treatment time.

FIG. 10 shows the measurement comparison charts of different concentration of nitrogen dioxide and different plasma treatment time.

FIG. 11 illustrates a comparison of the sensing response to ammonia and nitrogen dioxide of the gas sensing device of the invention in response to the plasma treatment time of carbon tetrafluoride (CF₄).

FIG. 12 illustrates a Raman analysis of the effect for the sensing layer (graphene) with different plasma treatment time.

FIG. 13 illustrates the top view of the gas sensing device according to another embodiment of the present invention.

FIG. 14 illustrates the sectional view of the gas sensing device according to another embodiment of the present invention.

### DETAILED DESCRIPTION OF THE INVENTION

In order to understand the technical features and practical efficacy of the present invention and to implement it in accordance with the contents of the specification, hereinafter, preferred embodiments of the present invention will be described in detail with reference to the accompanying drawings.

In order to overcome the problems to be solved and improve the sensing characteristics of gas sensing device, the invention provides a gas sensing device and its manufacturing method, which can improve the adsorption characteristics for gas selection ratio of sensing layer by plasma treatment for modifying the substrate.

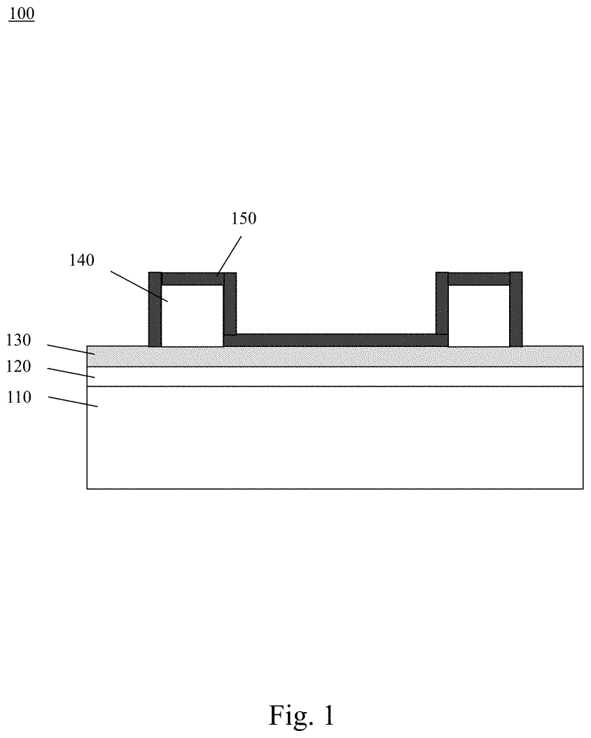

First, referring to FIG. 1, it illustrates a schematic diagram of a gas sensing device according to a preferred embodiment of the present invention. As shown in FIG. 1, the gas sensing device 100 comprises a substrate 110, an insulating layer 120, a plasma treatment layer 130, a metal electrode 140 and a sensing layer 150. Specifically, the insulation layer 120 is formed on the substrate 110, and the plasma treatment layer 130 is formed on the insulation layer 120. The metal electrode 140 is arranged (formed) on the plasma treatment layer 130, the sensing layer 150 is covered on the plasma treatment layer 130 and the metal electrode 140, and the sensing area is defined by the oxygen plasma.

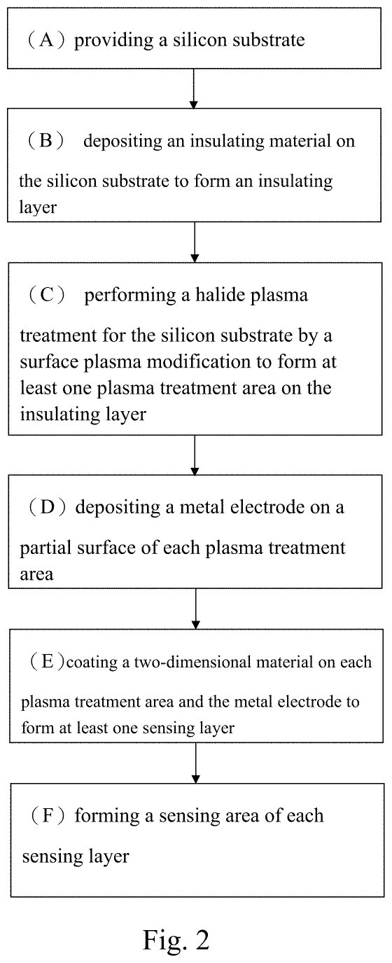

FIG. 2 illustrates a flow chart of the method for manufacturing the gas sensing device according to a preferred embodiment of the present invention. As shown in FIG. 2, the manufacturing method of the gas sensing device of the present embodiment includes the following steps: (A) providing a silicon substrate 110; (B) depositing an insulating material on the silicon substrate 110 to form an insulating layer 120; (C) performing a halide plasma treatment for the silicon substrate 110 and the insulating layer 120 for a period of time by a plasma surface modification to form at least one plasma treatment area (layer) 130 on the insulating layer 120; (D) depositing a metal electrode 140 on a partial surface of each plasma treatment area (layer) 130; (E) coating (covering) a two-dimensional material on each plasma treatment area (layer) 130 and the metal electrode 140 to form at least one sensing layer 150; and (F) forming a sensing area of each sensing layer 150.

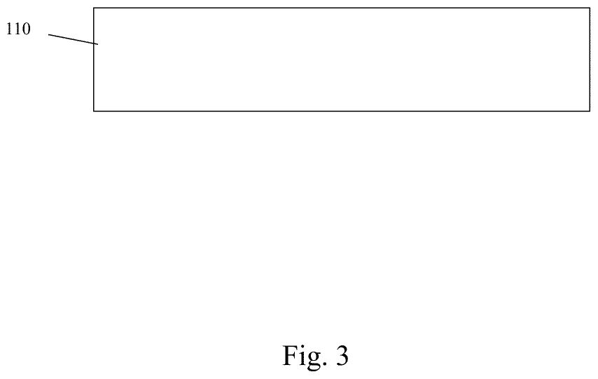

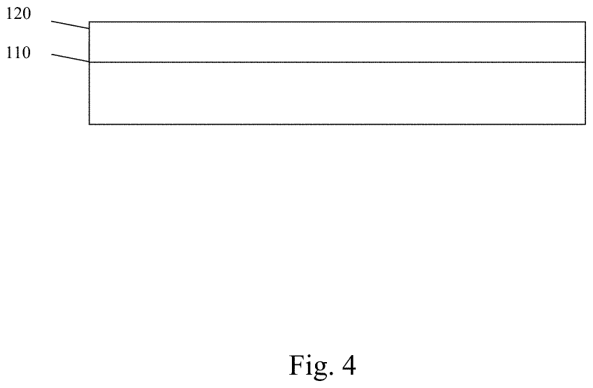

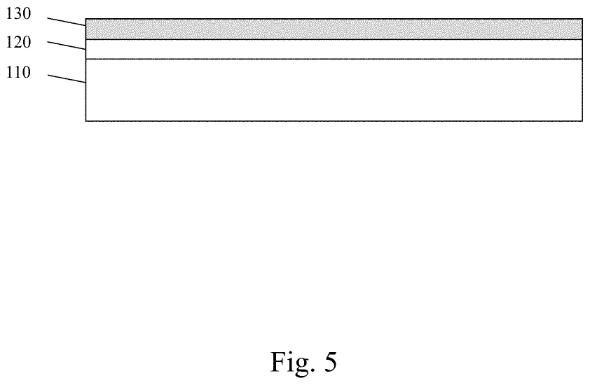

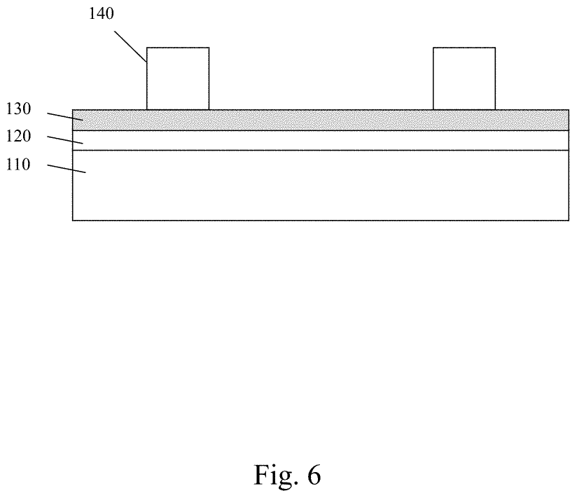

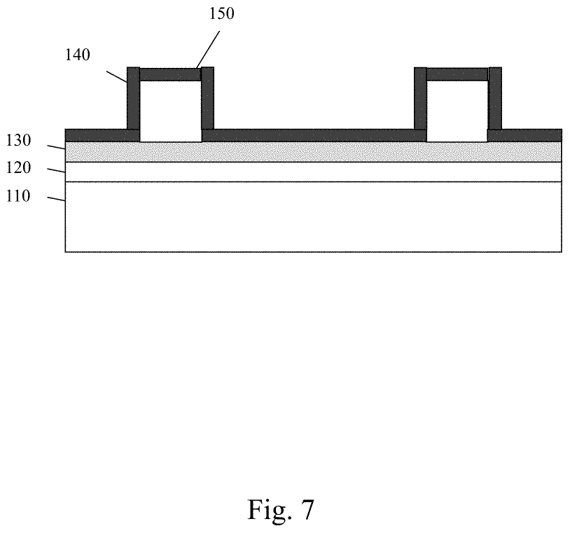

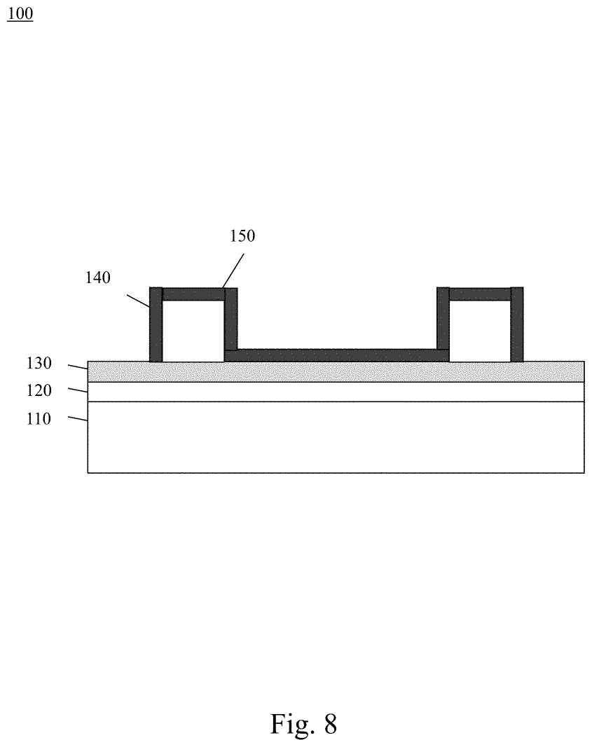

Next, please refer to FIG. 3 to FIG. 8. FIG. 3 shows a schematic diagram of the substrate of the preferred embodiment of the invention. FIG. 4 shows a schematic diagram of the insulation layer in a preferred embodiment of the invention. FIG. 5 shows a schematic diagram of the plasma treatment layer in the preferred embodiment of the invention. FIG. 6 shows a schematic diagram of the metal electrode structure in the preferred embodiment of the invention. FIG. 7 shows a schematic diagram of the sensing layer in the preferred embodiment of the invention. The gas sensing device according to a preferred embodiment of the present invention is shown in FIG. 8.

FIG. 3 to FIG. 8 further illustrate the manufacturing process of the gas sensing device of the present embodiment, which can be explained in accordance with the flow chart of the manufacturing method of FIG. 2. Firstly, as shown in FIG. 3, for the step (A) in the flow chart, a substrate 110, specifically a silicon substrate 110, is provided.

Then, as shown in FIG. 4, for the step (B) in the flow chart, an insulating material is deposited on the surface of the silicon substrate 110 to form an insulating layer 120 on the surface of the silicon substrate 110. The insulating material is silicon nitride (Si₃N₄).

As shown in FIG. 5, for the step (C) in the flow chart, a halide plasma treatment for the substrate 110 with the insulating layer 120 is performed for a period of time by a plasma surface modification to form a plasma treatment layer 130 on the insulating layer 120. The time period can be three minutes or six minutes, and a material of the halide can be carbon tetrafluoride (CF₄). However, the time period of the plasma treatment and the selection of halide material can be selected according to the different gases to be tested or the demand of user, and the present invention should not be limited accordingly.

As shown in FIG. 6, for the step (D) in the flow chart, a metal electrode 140 is deposited on the plasma treatment layer 130 by a photolithography process and a thin film deposition process. In this embodiment, the metal electrode 140 is configured in a two-end configuration and the spacing between the two electrodes 140 is 1000 to 2000 microns. In some embodiments, the pattern of the electrode can also be defined by a self-designed metal mask. The material of metal electrode 140 can be gold (Au), silver (Ag), copper (Cu), titanium (Ti) or their alloy, among which gold (Au) or titanium (Ti) is the better choice.

Furthermore, an adhesive layer (not shown) can be deposited at the junction of the plasma treatment layer 130 and the metal electrode 140.

As shown in FIG. 7, for the step (E) in the flow chart, the sensing layer 150 is coated (e.g. printed) on the metal electrode 140 and the plasma treatment layer 130. The sensing layer 150 can be made of two-dimensional material such as silicon, carbon nanotube, graphene or graphene oxide, among which the thin-film single-layer graphene is the better choice.

Finally, as shown in FIG. 8, for the step (F) in the flow chart, the remaining sensing layer (graphene) 150 is removed by oxygen plasma to define a sensing region, and therefore the gas sensing device 100 of the present embodiment is made.

In the gas sensing device of this embodiment, the silicon substrate 110 coated with silicon nitride (Si₃N₄) material will form the plasma treatment layer 130 with F—N electric dipole and negative charge accumulation on its surface by plasma treatment with carbon tetrafluoride (CF₄), resulting in an increase in the adsorption capacity of graphene as the sensing layer 150 for ammonia (NH₃) and a decrease in the adsorption capacity for nitrogen dioxide (NO₂).

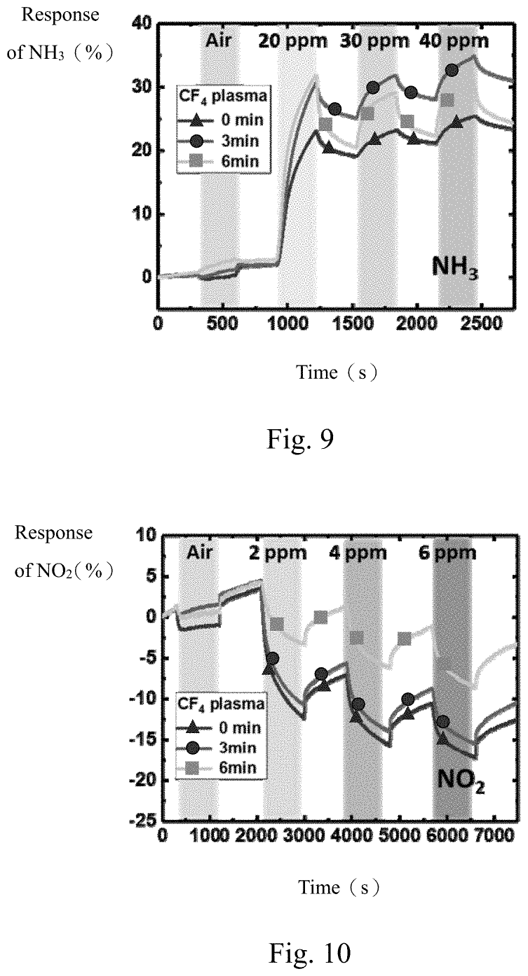

Further, referring to FIG. 9 and FIG. 10, FIG. 9 shows the measurement comparison charts based-on different concentration of ammonia and different plasma treatment time and FIG. 10 shows the measurement comparison charts of different concentration of nitrogen dioxide and different plasma treatment time. Firstly, as shown in FIG. 9, the sensitivity to ammonia response (velocity) of the gas sensing device increases with the increase of time at fixed ammonia concentration (20 ppm, 30 ppm and 40 ppm, respectively) by plasma treatment of carbon tetrafluoride (CF₄) for three minutes and six minutes. It can also be seen from FIG. 9 that the sensitivity of ammonia response (velocity) of the plasma-treated gas sensing device in this embodiment is obviously increased comparing with that of the sensor without plasma treatment, and the sensitivity of the response increases with the increase of plasma treatment time.

Relatively, as shown in FIG. 10, under the condition of fixed nitrogen dioxide concentration (2 ppm, 4 ppm and 6 ppm, respectively), the sensitivity to nitrogen dioxide response (velocity) of the gas sensing device decreases with the increase of time by plasma treatment of carbon tetrafluoride (CF₄) for three minutes and six minutes. It can also be seen from FIG. 10 that the sensitivity of the nitrogen dioxide response (velocity) of the plasma-treated gas sensing device in this embodiment is significantly reduced comparing with that of the sensor without plasma treatment, and the sensitivity of the response decreases with the increase of plasma treatment time.

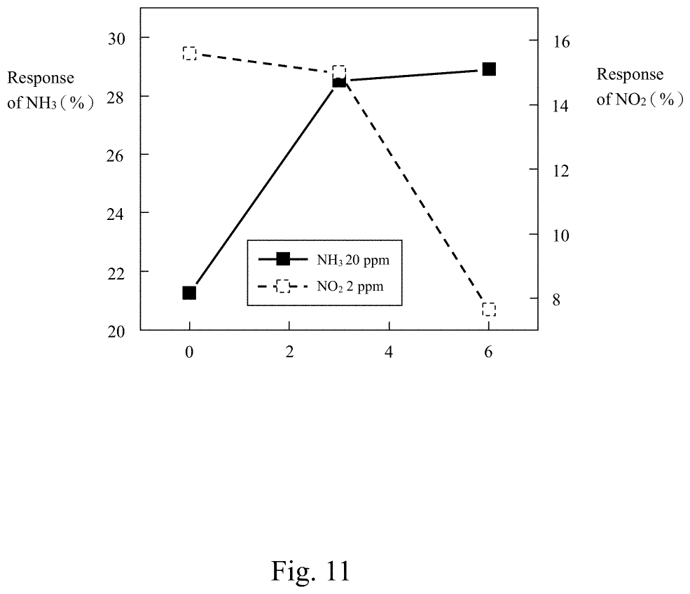

To sum up, referring to FIG. 11, it illustrates a comparison of the sensing response to ammonia and nitrogen dioxide of the gas sensing device of the invention in response to the plasma treatment time of carbon tetrafluoride (CF₄). As shown in FIG. 11, in the process of manufacturing the gas sensing device, the longer the plasma treatment time of carbon tetrafluoride (CF₄) is, the greater the response to ammonia (NH₃) and the smaller the response to nitrogen dioxide (NO₂) are. It can be concluded that the gas sensing device with the substrate having insulating layer for plasma treatment of carbon tetrafluoride (CF₄) by plasma surface modification has excellent ammonia gas binding ability and produces good reaction. The gas detected by the sensing layer is ammonia gas.

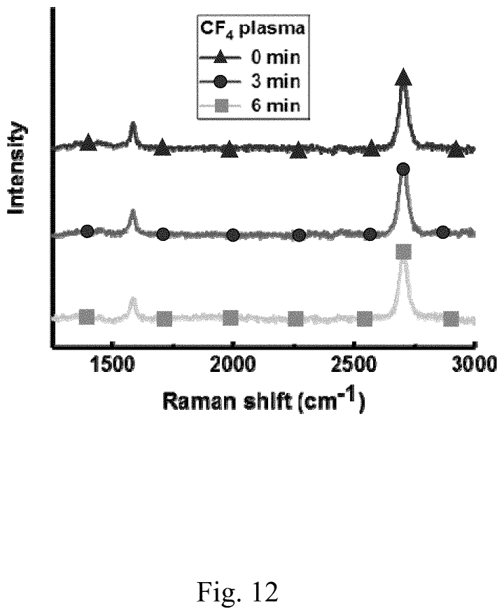

FIG. 12 is a Raman analysis of the effect for the sensing layer (graphene) with different plasma treatment time. According to the Raman analysis of FIG. 12, it can be seen that the plasma surface modified silicon substrate will not cause defects and structural changes in the graphene film structure of the sensing layer, regardless of the time of plasma modification.

Although only nitrogen dioxide (NO₂) and ammonia (NH₃) are mentioned in the present embodiment, according to the material for plasma surface modification of the present invention, gas molecules can be sensed by the gas sensing device of the present invention include NO, H₂ (hydrogen), O₂ (oxygen), CO₂, CO, NH₃ (ammonia), CH₃OCH₃ (dimethyl ether), C₃H₉O₃P (dimethyl methylphosphonate), C₂H₅OH (ethanol), CH₃OH (methanol), (CH₂)₄O (tetrahydrofuran), CHCl₃ (chloroform), H₂S (hydrogen sulfide) or C₃H₆O (acetone) which are selected according to user's demand, and the invention should not be limited to these.

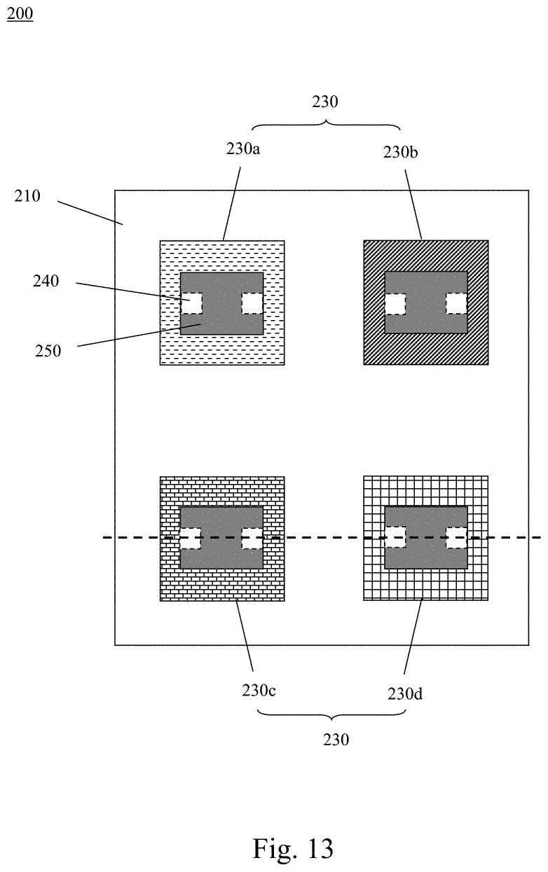

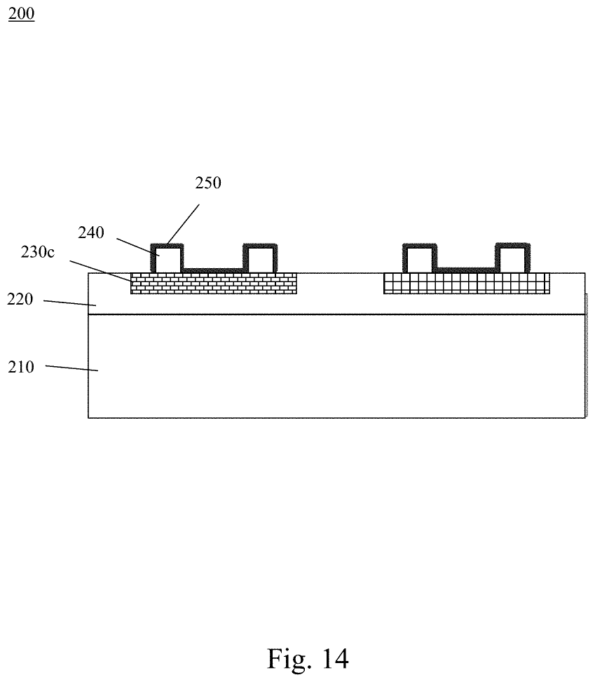

In addition, referring to FIG. 13 and FIG. 14, FIG. 13 illustrates the top view of the gas sensing device according to another embodiment of the present invention, and FIG. 14 illustrates the sectional view of the gas sensing device according to another embodiment of the present invention (along the dotted line of FIG. 13).

As shown in FIG. 13 and FIG. 14, a gas sensing device 200 according to another embodiment of the present invention comprises a substrate 210, an insulating layer 220 and an array plasma treatment layer 230. Specifically, the insulating layer 220 is formed on the substrate 210, the array plasma treatment layer 230 is formed on the insulating layer 220, and the array plasma treatment layer 230 has a plural plasma treatment area 230a, 230b, 230c and 230d. Each plasma treatment area 230a, 230b, 230c and 230d contains a metal electrode 240 which is formed (located) on the surface of each plasma treatment area 230a, 230b, 230c and 230d, and a sensing layer 250 is formed on the partial surface of each plasma treatment area 230a, 230b, 230c and 230d and the metal electrode 240.

Similarly, the following will further illustrate the manufacturing process of a gas sensing device according to another embodiment. First, a substrate 210, specifically a silicon substrate 210, is provided.

Subsequently, an insulating material is deposited on the surface of the silicon substrate 210 to form an insulating layer 220 on the surface of the silicon substrate 210. The insulating material is silicon nitride (Si₃N₄).

Next, in order to form an array of plasma treatment layer 230 on the insulating layer, a plasma treatment of halide or other material for the substrate 210 with an insulating layer for a period of time is carried out by plasma surface modification. It is should be noted that the array plasma treatment layer 230 has a plurality arrays of plasma treatment areas (zones) 230a, 230b, 230c and 230d, and each plasma treatment area 230a, 230b, 230c and 230d is separated from each other. Different halides (such as tetrafluorocarbon) or other materials are used for plasma surface modification for a period of time to form a plurality of plasma treatment area 230a, 230b, 230c and 230d with different materials used to sense various kinds of gas to be measured. In other words, the more plasma treatment areas 230a, 230b, 230c and 230d are, the more kinds of gas can be detected, and the total number of the plasma treatment areas 230a, 230b, 230c and 230d is larger than or equal to the kinds of gas to be measured.

In addition, although only 2*2 array arrangement is shown in FIG. 12, i.e., four plasma treatment areas (230a, 230b, 230c, 230d) formed by plasma modification with different halides or other materials for the identical substrate. In other embodiments, the number of plasma treatment areas (230a, 230b, 230c, 230d) can be adjusted according to user requirements. This invention is not to limit this number.

Furthermore, a metal electrode 240 is deposited on each plasma treatment area (230a, 230b, 230c, 230d) by a photolithography process and a deposition process. In this embodiment, the metal electrode 240 is configured in a two-end configuration and the spacing (distance) between the two electrodes is between 1000 and 2000 microns. In other embodiments, the pattern of the electrode can also be defined through a self-designed metal mask. Metal electrode 240 can be made of gold (Au), silver (Ag), copper (Cu), titanium (Ti) or their alloys, of which gold (Au) or titanium (Ti) is the better choice. Furthermore, an adhesive layer (not shown) can be deposited at the junction of each plasma treatment area (230a, 230b, 230c, 230d) with the metal electrode 240.

Furthermore, there is a sensing layer 250 covering (such as transfer printing) the metal electrode 240 and each plasma treatment area (230a, 230b, 230c, 230d). A two-dimensional material such as silicon, carbon nanotube, graphene or graphene oxide can be selected for the sensing layer 250, and a thin-film single layer graphene is the better choice.

Finally, oxygen plasma is used to remove the redundant sensing layer (graphene) 250 to define a sensing area of each sensing layer 250. The gas sensing device 200 with the array sensing areas (array of plasma treatment area 230a, 230b, 230c, 230d) can sense different kinds of gases according to this embodiment. For example, when the gas to be measured is a mixture of four gases, the mixture gases reacts with four different sensing regions in the gas sensing device 200, which changes the capacitance, resistance or electrical property of the sensing layer 250. Thus, the gas sensing device 200 of the present embodiment can simultaneously sense four different gases to achieve gas selectivity without additional gas separation system.

The gas sensing device of the above-mentioned two embodiments can be installed in various sensing apparatus or equipment according to their purposes, and the connection mode can be that the current/resistance data reader is connected with the electrode of the gas sensing device of the above-mentioned two embodiments, and the changes of the capacitance or resistance values of the sensing layer are detected for subsequent data processing.

In summary, after plasma doping and modification for the substrate of the gas sensing device of the invention, the sensing layer of the graphene film is transferred to the substrate and the electrode, and the response and selection ratio of gas to be measured for graphene are improved due to the sensing layer influenced by the modified substrate below. In addition, the invention also can plasma dope and modify the different materials at the same time on the substrate, so that the plural sensing layers are affected by the modified substrate below, and react with different gases to be measured, thus achieving the characteristics of a single sensing device to detect various gases to be measured.

As is understood by a person skilled in the art, the foregoing preferred embodiments of the present invention are illustrated of the present invention rather than limiting of the present invention. It is intended to cover various modifications and similar arrangements included within the spirit and scope of the appended claims, the scope of which should be accorded the broadest interpretation so as to encompass all such modifications and similar structure. While the preferred embodiment of the invention has been illustrated and described, it will be appreciated that various changes can be made therein without departing from the spirit and scope of the invention.

What is claimed is:

1. A gas sensing device, comprising:
a substrate;
an insulating layer formed on said substrate;
a plasma treatment layer formed on said insulation layer;
a metal electrode disposed on said plasma treatment layer; and
a sensing layer formed on a surface of said plasma treatment layer and said metal electrode.

2. The device of claim 1, wherein a material of said plasma treatment layer is halide.

3. The device of claim 2, wherein said halide is carbon tetrafluoride (CF₄).

4. The device of claim 1, wherein a material of said sensing layer is a two-dimensional material.

5. The device of claim 4, wherein said two-dimensional material is graphene.

6. The device of claim 1, wherein an adhesive layer is deposited at a junction of said plasma treatment layer and said metal electrode.

7. A gas sensing device, comprising:
a substrate;
an insulating layer formed on said substrate;
an array plasma treatment layer including a plurality of plasma treatment areas,
wherein said array plasma treatment layer is formed on said insulation layer, each of said plurality of plasma treatment areas includes:
a metal electrode formed on a surface of said each of said plurality of plasma treatment areas; and
a sensing layer formed on a surface of said each of said plurality of plasma treatment areas and said metal electrode.

8. The device of claim 7, wherein a material of said plural of plasma treatment areas is halide.

9. The device of claim 8, wherein said halide is carbon tetrafluoride (CF₄).

10. The device of claim 7, wherein a material of said sensing layer is a two-dimensional material.

11. The device of claim 10, wherein said two-dimensional material is graphene.

12. The device of claim 7, wherein an adhesive layer is deposited at a junction of said each of said plural of plasma treatment areas and said metal electrode.

13. A manufacturing method of a gas sensing device, comprising:
(A) providing a substrate;
(B) depositing an insulating material on said substrate to form an insulating layer;
(C) performing a halide plasma treatment for said substrate for a period of time by a plasma surface modification to form at least one plasma treatment area on said insulating layer;
(D) depositing a metal electrode on a partial surface of each said at least one plasma treatment area;
(E) coating a two-dimensional material on said each said at least one and said metal electrode to form at least one sensing layer; and
(F) forming a sensing area of each said at least one sensing layer.

14. The method of claim 13, wherein a material of said insulating layer in said step (B) 1S Si₃N₄.

15. The method of claim 13, wherein said halide in said step (C) is carbon tetrafluoride (CF₄).

16. The method of claim 13, wherein said period of time in said step (C) is three minutes or six minutes.

17. The method of claim 13, wherein, in said step (D), an adhesive layer is deposited at a junction of said each of said at least one plasma treatment area and said metal electrode.

18. The method of claim 13, wherein, in said step (E), said two-dimensional material is graphene.

19. The method of claim 13, wherein, in said step (F), said sensing area is defined by an oxygen plasma
